(19) United States
(12) Patent Application Publication
(10) Pub. No.: US 2017/0172414 A1
(43) Pub. Date: Jun. 22, 2017

(54) Multiple Patient EEG Monitoring

(71) Applicant: Persyst Development Corporation, San Diego, CA (US)

(72) Inventors: Nicolas Nierenberg, La Jolla, CA (US); Scott B. Wilson, Del Mar, CA (US); Mark L. Scheuer, Wexford, PA (US); Andrey Pirozhenko, San Diego, CA (US); Michael Guess, Prescott, AZ (US)

(73) Assignee: Persyst Development Corporation, San Diego, CA (US)

(21) Appl. No.: 15/449,944

(22) Filed: Mar. 4, 2017

Related U.S. Application Data
(63) Continuation of application No. 13/831,609, filed on Mar. 15, 2013.
Application No. 13/831,609 is a continuation-in-part of application No. 13/620,855, filed on Sep. 15, 2012, now abandoned.
(60) Provisional application No. 61/536,236, filed on Sep. 19, 2011.

(51) Int. Cl. A61B 5/00; A61B 5/04; A61B 5/048

(52) U.S. Cl. CPC A61B 5/0006; A61B 5/048; A61B 5/04012; A61B 5/4094; A61B 5/7445; A61B 5/7203; A61B 5/002

(57) ABSTRACT

A system and method for multiple EEG acquisition and monitoring is disclosed herein. The system includes multiple EEG machines connected to a central station over a network. Each EEG machine of the multiple EEG machines has a separate cell on a screen page to allow for an operator to monitor each of the EEG machines.

### CROSS REFERENCE TO RELATED APPLICATION

The Present Application is a continuation application of U.S. patent application Ser. No. 13/831,609, filed on Mar. 15, 2013, which is a continuation-in-part application of U.S. patent application Ser. No. 13/620,855, filed on Sep. 15, 2012, which claims priority to U.S. Provisional Patent Application No. 61/536,236, filed on Sep. 19, 2011, now expired, all of which are hereby incorporated by reference in their entireties.

### STATEMENT REGARDING FEDERALLY SPONSORED RESEARCH OR DEVELOPMENT

Not Applicable

### BACKGROUND OF THE INVENTION

Field of the Invention

The present invention generally relates to a method and system for displaying EEG data. More specifically, the present invention relates to a method and system for acquiring and monitoring multiple EEG machines.

Description of the Related Art

An electroencephalogram (“EEG”) is a diagnostic tool that measures and records the electrical activity of a person's brain in order to evaluate cerebral functions. Multiple electrodes are attached to a person's head and connected to a machine by wires. The machine amplifies the signals and records the electrical activity of a person's brain. The electrical activity is produced by the summation of neural activity across a plurality of neurons. These neurons generate small electric voltage fields. The aggregate of these electric voltage fields create an electrical reading which electrodes on the person's head are able to detect and record. An EEG is a superposition of multiple simpler signals. In a normal adult, the amplitude of an EEG signal typically ranges from 1 micro-Volt to 100 micro-Volts, and the EEG signal is approximately 10 to 20 milli-Volts when measured with subdural electrodes. The monitoring of the amplitude and temporal dynamics of the electrical signals provides information about the underlying neural activity and medical conditions of the person.

An EEG is performed to: diagnose epilepsy; verify problems with loss of consciousness or dementia; verify brain activity for a person in a coma; study sleep disorders, monitor brain activity during surgery, and additional physical problems.

Multiple electrodes (typically 17-21, however there are standard positions for at least 70) are attached to a person's head during an EEG. The electrodes are referenced by the position of the electrode in relation to a lobe or area of a person's brain. The references are as follows: F=frontal; Fp=frontopolar; T=temporal; C=central; P=parietal; O=occipital; and A=auricular (ear electrode). Numerals are used to further narrow the position and “z” points relate to electrode sites in the midline of a person's head. An electrocardiogram (“EKG”) may also appear on an EEG display.

The EEG records brain waves from different amplifiers using various combinations of electrodes called montages. Montages are generally created to provide a clear picture of the spatial distribution of the EEG across the cortex. A montage is an electrical map obtained from a spatial array of recording electrodes and preferably refers to a particular combination of electrodes examined at a particular point in time.

In bipolar montages, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode.

Reference montages are good for determining the true amplitude and morphology of a waveform. For temporal electrodes, CZ is usually a good scalp reference.

Being able to locate the origin of electrical activity (“localization”) is critical to being able to analyze the EEG. Localization of normal or abnormal brain waves in bipolar montages is usually accomplished by identifying “phase reversal,” a deflection of the two channels within a chain pointing to opposite directions. In a referential montage, all channels may show deflections in the same direction. If the electrical activity at the active electrodes is positive when compared to the activity at the reference electrode, the deflection will be downward. Electrodes where the electrical activity is the same as at the reference electrode will not show any deflection. In general, the electrode with the largest upward deflection represents the maximum negative activity in a referential montage.

Some patterns indicate a tendency toward seizures in a person. A physician may refer to these waves as “epileptiform abnormalities” or “epilepsy waves.” These include spikes, sharp waves, and spike-and-wave discharges. Spikes and sharp waves in a specific area of the brain, such as the left temporal lobe, indicate that partial seizures might possibly come from that area. Primary generalized epilepsy, on the other hand, is suggested by spike-and-wave discharges that are widely spread over both hemispheres of the brain, especially if they begin in both hemispheres at the same time.

There are several types of brain waves: alpha waves, beta waves, delta wave, theta waves and gamma waves. Alpha waves have a frequency of 8 to 12 Hertz (“Hz”). Alpha waves are normally found when a person is relaxed or in a waking state when a person's eyes are closed but the person is mentally alert. Alpha waves cease when a person's eyes are open or the person is concentrating. Beta waves have a frequency of 13 Hz to 30 Hz. Beta waves are normally found when a person is alert, thinking, agitated, or has taken high doses of certain medicines. Delta waves have a frequency of less than 3 Hz. Delta waves are normally found only when a person is asleep (non-REM or dreamless sleep) or the person is a young child. Theta waves have a frequency of 4 Hz to 7 Hz. Theta waves are normally found only when the person is asleep (dream or REM sleep) or the person is a young child. Gamma waves have a frequency of 30 Hz to 100 Hz. Gamma waves are normally found during higher mental activity and motor functions.

The following definitions are used herein.

“Amplitude” refers to the vertical distance measured from the trough to the maximal peak (negative or positive). It expresses information about the size of the neuron population and its activation synchrony during the component generation.

The term “analogue to digital conversion” refers to when an analogue signal is converted into a digital signal which can then be stored in a computer for further processing. Analogue signals are “real world” signals (e.g., physiological signals such as electroencephalogram, electrocardiogram or electrooculogram). In order for them to be stored and manipulated by a computer, these signals must be converted into a discrete digital form the computer can understand.

“Artifacts” are electrical signals detected along the scalp by an EEG, but that originate from non-cerebral origin. There are patient related artifacts (e.g., movement, sweating, ECG, eye movements) and technical artifacts (50/60 Hz artifact, cable movements, electrode paste-related).

The term “differential amplifier” refers to the key to electrophysiological equipment. It magnifies the difference between two inputs (one amplifier per pair of electrodes).

“Duration” is the time interval from the beginning of the voltage change to its return to the baseline. It is also a measurement of the synchronous activation of neurons involved in the component generation.

“Electrode” refers to a conductor used to establish electrical contact with a nonmetallic part of a circuit. EEG electrodes are small metal discs usually made of stainless steel, tin, gold or silver covered with a silver chloride coating. They are placed on the scalp in special positions.

“Electrode gel” acts as a malleable extension of the electrode, so that the movement of the electrodes leads is less likely to produce artifacts. The gel maximizes skin contact and allows for a low-resistance recording through the skin.

The term “electrode positioning” (10/20 system) refers to the standardized placement of scalp electrodes for a classical EEG recording. The essence of this system is the distance in percentages of the 10/20 range between Nasion-Inion and fixed points. These points are marked as the Frontal pole (Fp), Central (C), Parietal (P), occipital (O), and Temporal (T). The midline electrodes are marked with a subscript z, which stands for zero. The odd numbers are used as subscript for points over the left hemisphere, and even numbers over the right

“Electroencephalogram” or “EEG” refers to the tracing of brain waves, by recording the electrical activity of the brain from the scalp, made by an electroencephalograph.

“Electroencephalograph” refers to an apparatus for detecting and recording brain waves (also called encephalograph).

“Epileptiform” refers to resembling that of epilepsy.

“Filtering” refers to a process that removes unwanted frequencies from a signal.

“Filters” are devices that alter the frequency composition of the signal.

“Montage” means the placement of the electrodes. The EEG can be monitored with either a bipolar montage or a referential one. Bipolar means that there are two electrodes per one channel, so there is a reference electrode for each channel. The referential montage means that there is a common reference electrode for all the channels.

“Morphology” refers to the shape of the waveform. The shape of a wave or an EEG pattern is determined by the frequencies that combine to make up the waveform and by their phase and voltage relationships. Wave patterns can be described as being: “Monomorphic”. Distinct EEG activity appearing to be composed of one dominant activity. “Polymorphic”. distinct EEG activity composed of multiple frequencies that combine to form a complex waveform. “Sinusoidal”. Waves resembling sine waves. Monomorphic activity usually is sinusoidal. “Transient”. An isolated wave or pattern that is distinctly different from background activity.

“Spike” refers to a transient with a pointed peak and a duration from 20 to under 70 msec.

The term “sharp wave” refers to a transient with a pointed peak and duration of 70-200 msec.

The term “neural network algorithms” refers to algorithms that identify sharp transients that have a high probability of being epileptiform abnormalities.

“Noise” refers to any unwanted signal that modifies the desired signal. It can have multiple sources.

“Periodicity” refers to the distribution of patterns or elements in time (e.g., the appearance of a particular EEG activity at more or less regular intervals). The activity may be generalized, focal or lateralized.

An EEG epoch is an amplitude of a EEG signal as a function of time and frequency.

There is a need to be able to monitor multiple EEG machines from a single site since a facility such as a hospital, may only have one technician skilled in reading EEG recordings.

### BRIEF SUMMARY OF THE INVENTION

The present invention provides for monitoring and analysis of EEG recordings for multiple EEG machines at a central location. Although, multiple EEG machines are monitored, the present invention allows for each EEG recording to be monitored independently and without affecting the other EEG recordings.

One aspect of the present invention is a method for monitoring multiple EEG machines and acquiring EEG recordings for each of the EEG machines. The method includes monitoring a plurality of EEG machines from a central station. Each of the plurality of EEG machines comprises a plurality of electrodes, an amplifier and processor. The central station is in communication with each of the plurality of EEG machine over a network. The method also includes acquiring EEG signals from each active EEG machine of the plurality of EEG machines. The method also includes processing the EEG signals to generate processed EEG recordings for each of the active EEG machines of the plurality of EEG machines. The method also includes displaying each of the processed EEG recordings for each of the active EEG machines of the plurality of EEG machines in a separate cell on a display page at a client device.

Another aspect of the present invention is a system for monitoring multiple EEG machines and acquiring EEG recording from each EEG machine. The system includes multiple EEG machines, a network, and a client site. Each of EEG machines comprises a plurality of electrodes for generating a plurality of EEG signals. The client site comprises a processing engine, a database and a display monitor. The processing engine is configured to process each of the plurality of EEG signals from each of the EEG machines to generate a plurality of processed EEG recordings. The processing engine is configured to display each of the EEG recordings in a separate cell in a display page for display on the display monitor.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a block diagram of a system for monitoring multiple EEG machines and acquiring EEG recordings for each of the EEG machines.

FIG. 2 is an illustration of a multiple cell display page for monitoring multiple EEG machines.

FIG. 2A an enlarge view of dashed line box 2A of FIG. 2.

FIG. 3 is an illustration of a single cell display page for monitoring multiple EEG machines.

FIG. 3A an enlarge view of dashed line box 3A of FIG. 3.

FIG. 3B an enlarge view of dashed line box 3B of FIG. 3.

FIG. 4 is a block diagram of a system for monitoring multiple EEG machines and acquiring EEG recordings for each of the EEG machines.

FIG. 5 is a flow chart for a method for monitoring multiple EEG machines and acquiring EEG recordings for each of the EEG machines.

### DETAILED DESCRIPTION OF THE INVENTION

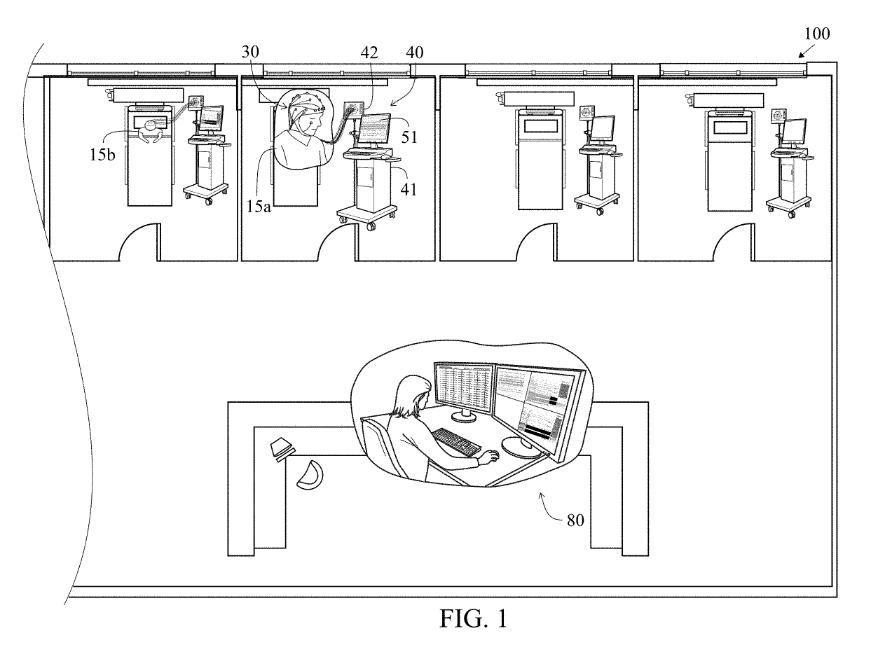

As shown in FIG. 1, a system for monitoring multiple EEG machines and acquiring EEG recordings for each of the EEG machines is generally designated 100. The system 100 comprises a plurality of EEG machines 40, a network and a central station 80. Preferably, each of the EEG machines 40 is located within a single facility along with the central station 80. Alternatively, each of the EEG machines 40 is located at various facilities and connected to the central station over the network.

The system 100 allows for monitoring multiple EEG machines 40 and acquiring EEG recordings for each active EEG machine at the central station 80.

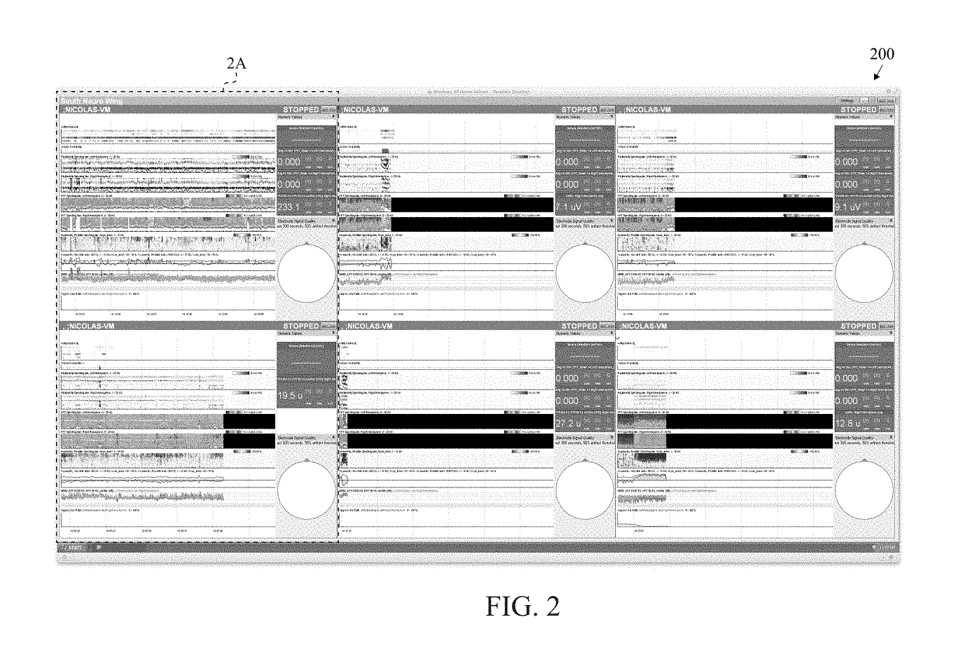

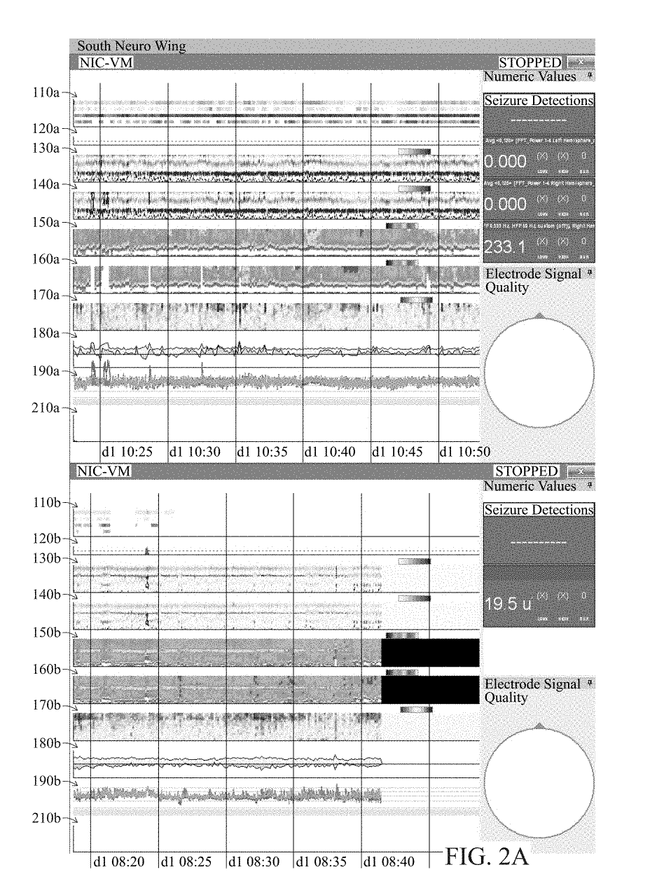

Each of the EEG machines 40 preferably comprises a plurality of electrodes 30, an amplifier 42, a processor 41 and a display 51. FIG. 2 illustrates a system 20 for a user interface for automated artifact filtering for an EEG. A patient 15 wears an electrode cap 31, consisting of a plurality of electrodes 35a-35c, attached to the patient's head with wires 38 from the electrodes 35 connected to an EEG machine component 40 which consists of an amplifier 42 for amplifying the signal to a computer 41 with a processor, which is used to analyze the signals from the electrodes 35 and create an EEG recording 51, which can be viewed on a display 50. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is analyzed for display.

A client device at the central station 80 preferably has an EEG processing engine to display each of the EEG recordings for each of the EEG machines 40 in a separate cell in a display page for display on the display monitor at the central station 80. The processing engine is configured to preferably display a plurality of trends for each of the EEG recordings in each of the separate cells in the display page. The processing engine is configured to preferably assign a separate cell to a newly active EEG machine. The processing engine is configured to preferably allow for switching between multiple modes. The processing engine is configured to preferably update each of the separate cells in real-time. The processing engine is configured to preferably permit one-click to open an active EEG recording and occupy the entire display page with the single separate cell. The processing engine is configured to preferably assign a user interface. The processing engine is configured to preferably change a parameter of a plurality of parameters of one cell without affecting the plurality of parameters of any of the other cells on the display page. The processing engine is configured to preferably move through different open EEG recordings on the display page, move to a partial EEG recording. The processing engine is configured to preferably allow for review of an entire EEG record on the display page.

An additional description of analyzing EEG recordings is set forth in

Wilson et al., U.S. patent application Ser. No. 13/620,855, filed on Sep. 15, 2012, for a Method And System For Analyzing An EEG Recording, which is hereby incorporated by reference in its entirety.

A patient has a plurality of electrodes attached to the patient's head with wires from the electrodes connected to an amplifier for amplifying the signal to a processor, which is used to analyze the signals from the electrodes and create an EEG recording. The brain produces different signals at different points on a patient's head. Multiple electrodes are positioned on a patient's head. The CZ site is in the center. The number of electrodes determines the number of channels for an EEG. A greater number of channels produce a more detailed representation of a patient's brain activity. Preferably, each amplifier 42 of an EEG machine component 40 corresponds to two electrodes 35 attached to a head of the patient 15. The output from an EEG machine component 40 is the difference in electrical activity detected by the two electrodes. The placement of each electrode is critical for an EEG report since the closer the electrode pairs are to each other, the less difference in the brainwaves that are recorded by the EEG machine component 40. A more thorough description of an electrode utilized with the present invention is detailed in Wilson et al., U.S. Pat. No. 8,112,141 for a Method And Device For Quick Press On EEG Electrode, which is hereby incorporated by reference in its entirety.

FIG. 2 is an illustration of a display page 200. As shown in FIG. 2, this display page 20 has six separate cells that each represent a separate EEG machine 40 connected to the client device over a network. Each separate cell can display different trends for each EEG recording from each EEG machine. As shown in FIG. 2A, a separate cell of the display page 200 displays multiple trends for a EEG recording.

As shown in FIG. 2A, the multiple trends can be different for each separate cell. An artifact intensity trend 110a is shown as a series of horizontal lines. The plurality of horizontal lines shown comprises a horizontal line for a muscle artifact, a horizontal line for a chewing artifact, a horizontal line for a vertical eye movement artifact, and a horizontal line for a lateral eye movement artifact. Those skilled in the pertinent art will recognize that more or less horizontal lines may be used without departing from the scope and spirit of the present invention.

Also shown in FIG. 2A for the upper separate cell are a seizure probability trend 120a, a rhythmicity spectrogram, left hemisphere trend 130a, a rhythmicity spectrogram, right hemisphere trend 140a, a FFT spectrogram left hemisphere trend 150a, a FFT spectrogram right hemisphere trend 160a, an asymmetry relative spectrogram trend 170a, a asymmetry absolute index trend 180a, an aEEG trend 190a, and a suppression ration, left hemisphere and right hemisphere trend 210a.

Also shown in FIG. 2A are trends for a different separate cell, which include a seizure probability trend 120b, a rhythmicity spectrogram, left hemisphere trend 130b, a rhythmicity spectrogram, right hemisphere trend 140b, a FFT spectrogram left hemisphere trend 150b, a FFT spectrogram right hemisphere trend 160b, an asymmetry relative spectrogram trend 170b, a asymmetry absolute index trend 180b, an aEEG trend 190b, and a suppression ration, left hemisphere and right hemisphere trend 210b.

The display page 200 illustrates that the artifact intensity trend 110a for the upper separate cell has a much great amount of artifacts than the artifact intensity trend 110b of the lower separate cell of the display page 200.

Rhythmicity spectrograms allow one to see the evolution of seizures in a single image. The rhythmicity spectrogram measures the amount of rhythmicity which is present at each frequency in an EEG record.

The seizure probability trend shows a calculated probability of seizure activity over time. The seizure probability trend shows the duration of detected seizures, and also suggests areas of the record that may fall below the seizure detection cutoff, but are still of interest for review. The seizure probability trend when displayed along with other trends, provides a comprehensive view of quantitative changes in an EEG.

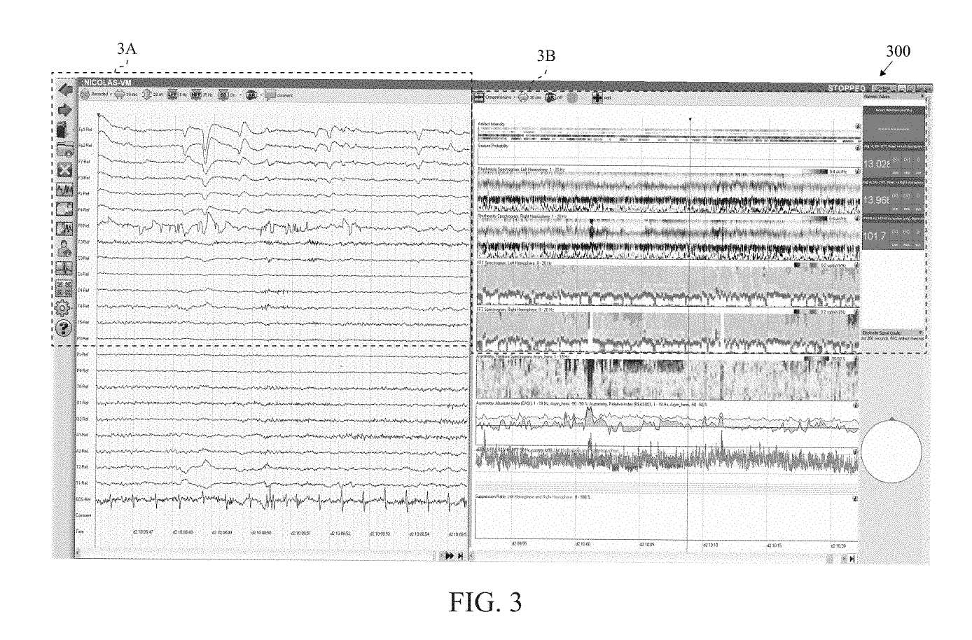

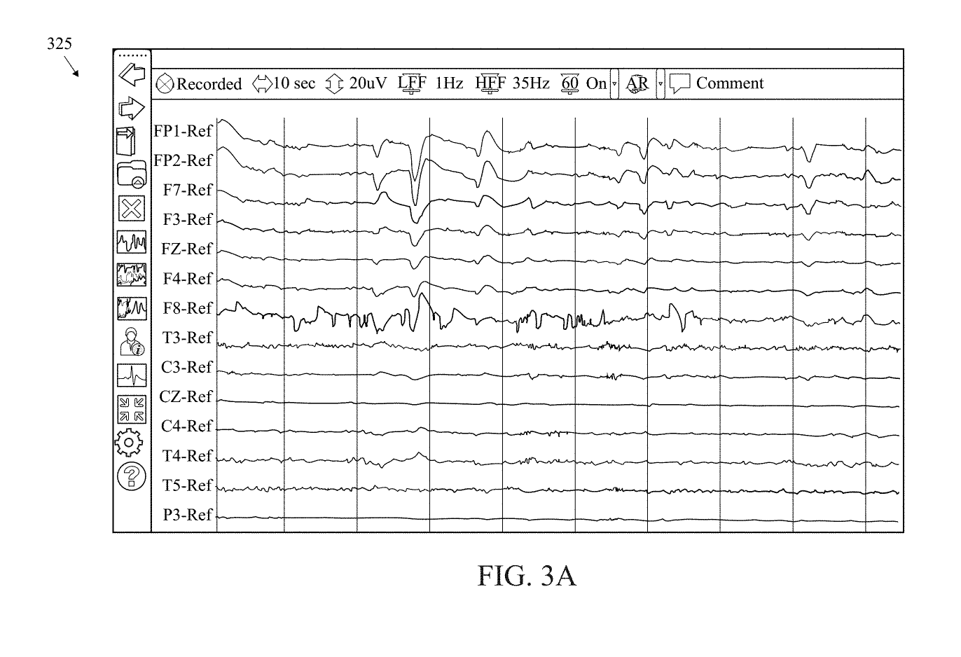

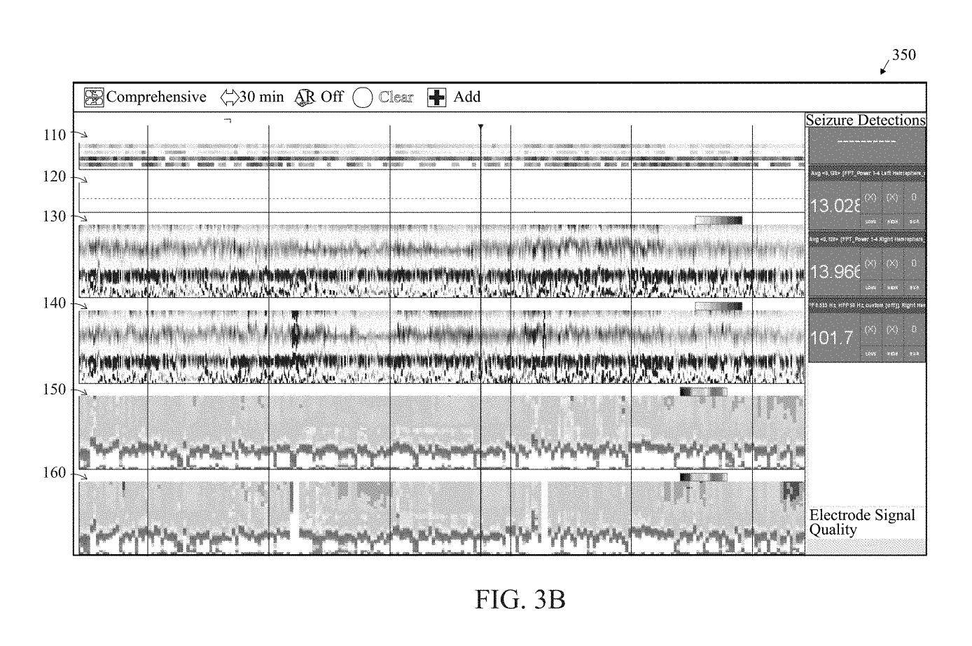

A full display page view of a single cell is shown in FIG. 3. The processing engine allows for a one-click mechanism to move from multiple separate cells on a display page to a full screen view of a single separate cell. Thus, as shown in FIGS. 3A and 3B, a real-time EEG recording and trends for a single separate cell are displayed on a single display page.

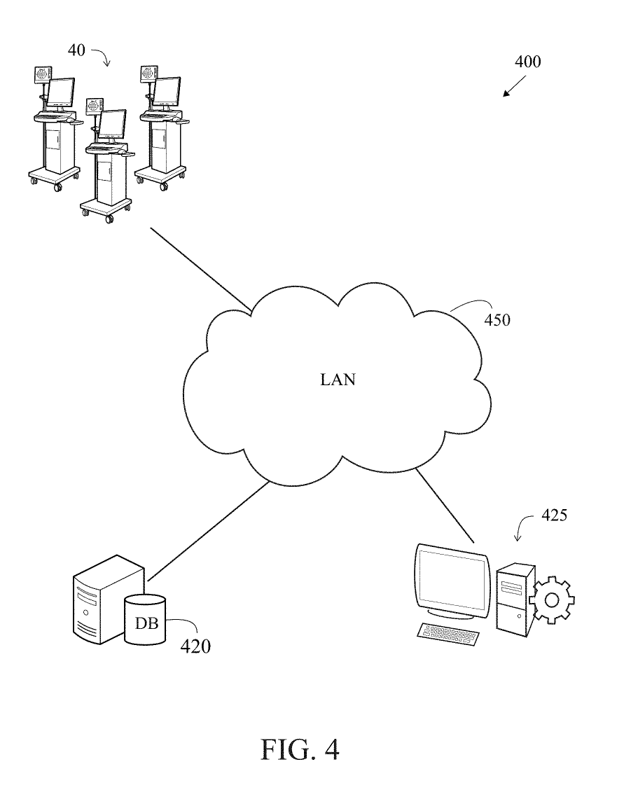

A system 400 for monitoring multiple EEG machines and acquiring EEG recording from each EEG machine is shown in FIG. 4. The system 100 comprises a plurality of EEG machines 40, a network 450 (preferably a local area network), a client site 425 and a database 420 for storing EEG recordings. Preferably, each of the EEG machines 40 is located within a single facility along with the central station 80. Alternatively, each of the EEG machines 40 is located at various facilities and connected to the central station over the network.

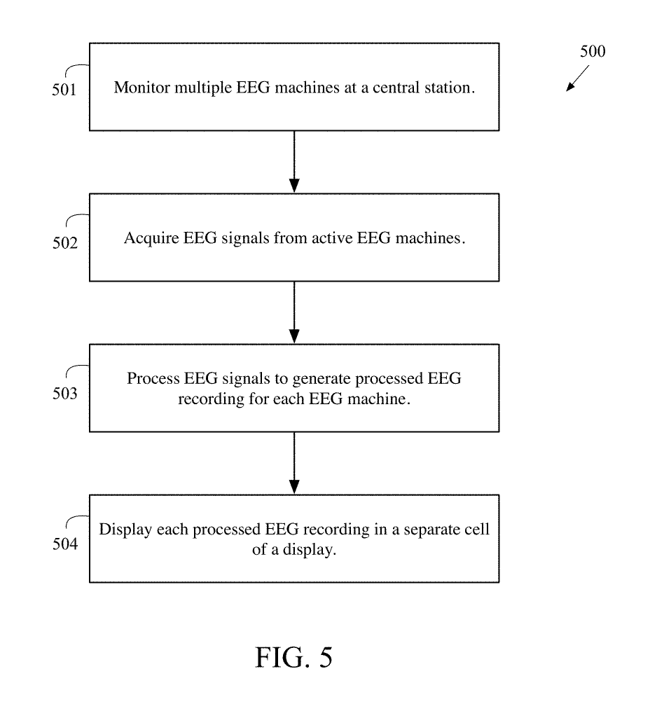

A method 500 for monitoring multiple EEG machines and acquiring EEG recordings for each of the EEG machines is shown in FIG. 5. At block 501, multiple EEG machines are monitored from a central station. Each of the plurality of EEG machines comprises a plurality of electrodes, an amplifier and processor. The central station is in communication with each of the plurality of EEG machine over a network. At block 502, EEG signals are acquired from each active EEG machine of the plurality of EEG machines. At block 503, the EEG signals are processed to generate processed EEG recordings for each of the active EEG machines of the plurality of EEG machines. At block 504, each of the processed EEG recordings for each of the active EEG machines of the plurality of EEG machines is displayed in a separate cell on a display page at a display monitor at a client device.

The EEG is optimized for automated artifact filtering. The EEG recordings are then processed using neural network algorithms to generate a processed EEG recording, which is analyzed for display. During acquisition of the EEG recording, a processing engine performs continuous analysis of the EEG waveforms and determines the presence of most types of electrode artifact on a channel-by-channel basis. Much like a human reader, the processing engine detects artifact by analyzing multiple features of the EEG traces. The preferred artifact detection is independent of impedance checking. During acquisition the processing monitors the incoming channels looking for electrode artifacts. When artifacts are detected they are automatically removed from the seizure detection process and optionally removed from the trending display. This results in much a much higher level of seizure detection accuracy and easier to read trends than in previous generation products.

Algorithms for removing artifact from EEG typically use Blind Source Separation (BSS) algorithms like CCA (canonical correlation analysis) and ICA (Independent Component Analysis) to transform the signals from a set of channels into a set of component waves or “sources.”

In one example an algorithm called BSS-CCA is used to remove the effects of muscle activity from the EEG. Using the algorithm on the recorded montage will frequently not produce optimal results. In this case it is generally optimal to use a montage where the reference electrode is one of the vertex electrodes such as CZ in the international 10-20 standard. In this algorithm the recorded montage would first be transformed into a CZ reference montage prior to artifact removal. In the event that the signal at CZ indicates that it is not the best choice then the algorithm would go down a list of possible reference electrodes in order to find one that is suitable.

It is possible to perform BSS-CCA directly on the user-selected montage. However this has two issues. First this requires doing an expensive artifact removal process on each montage selected for viewing by the user. Second the artifact removal will vary from one montage to another, and will only be optimal when a user selects a referential montage using the optimal reference. Since a montage that is required for reviewing an EEG is frequently not the same as the one that is optimal for removing artifact this is not a good solution.

An additional description of analyzing EEG recordings is set forth in Wilson et al., U.S. patent application Ser. No. 13/684,469, filed on Nov. 23, 2012, for a User Interface For Artifact Removal In An EEG, which is hereby incorporated by reference in its entirety. An additional description of analyzing EEG recordings is set forth in Wilson et al., U.S. patent application Ser. No. 13/684,556, filed on Nov. 25, 2012, for a Method And System For Detecting And Removing EEG Artifacts, which is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention:

1. A system for monitoring multiple electroencephalogram (EEG) machines and acquiring EEG recording from each EEG machine, the system comprising:
a plurality of EEG machines, each of the plurality of EEG machine comprising a plurality of electrodes for generating a plurality of EEG signals, each of the plurality of EEG machines in a separate room;
a network;
a client site comprising a processing engine, a database and a display monitor, the processing engine configured to process each of the plurality of EEG signals from each of the EEG machines to generate a plurality of processed EEG recordings;
wherein the processing engine is configured to display each of the EEG recordings in a separate cell in a display page for display on the display monitor;
wherein the processing engine is configured to monitor each channel for electrode artifacts during acquisition of the EEG signals and remove the electrode artifacts to provide a higher level of seizure detection accuracy than without removal of the electrode artifacts;
wherein the processing engine is configured to detect artifacts in the EEG signals by analyzing a plurality of features;
wherein an artifact intensity is illustrated in each of the plurality of separate cells, the artifact intensity shown as a series of horizontal lines comprising a horizontal line for a muscle artifact, a horizontal line for a chewing artifact, a horizontal line for a vertical eye movement artifact, and a horizontal line for a lateral eye movement artifact;
wherein the plurality of trends comprises a seizure probability trend showing a calculated probability of seizure activity over a predetermined time, a rhythmicity spectrogram measuring the amount of rhythmicity which is present at each frequency in an EEG record, a left hemisphere trend, a right hemisphere trend, a fast Fourier transformation (FFT) spectrogram left hemisphere trend, a FFT spectrogram right hemisphere trend, an asymmetry relative spectrogram trend, and an ambulatory EEG (aEEG).

2. The system according to claim 1 wherein a plurality of trends is displayed for each of the EEG recordings in each of the separate cells in the display page.

3. The system according to claim 1 wherein the processing engine is configured to assign a separate cell to a newly active EEG machine.

4. The system according to claim 1 wherein the processing engine is configured to allow for switching between multiple modes.

5. The system according to claim 1 wherein the processing engine is configured to update each of the separate cells in real-time.

6. The system according to claim 1 wherein the processing engine is configured to permit one-click to open an active EEG recording and occupy the entire display page with the single separate cell.

7. The system according to claim 1 wherein the processing engine is configured to assign a user interface.

8. The system according to claim 1 wherein the processing engine is configured to change a parameter of a plurality of parameters of one cell without affecting the plurality of parameters of any of the other cells on the display page.

9. The system according to claim 1 wherein the processing engine is configured to move through different open EEG recordings on the display page, move to a partial EEG recording, and permit one-click to open an active EEG recording and occupy the entire display page with the single separate cell.

10. The system according to claim 1 wherein the processing engine is configured to allow for review of an entire EEG record on the display page.

11. The system according to claim 1 wherein each of the EEG machines of the plurality of EEG machines further comprises a processor connected to the plurality of electrodes to generate an EEG recording from the plurality of EEG signals and a display connected to the processor for displaying an EEG recording in proximity to the patient.

12. A method for monitoring multiple electroencephalogram (EEG) machines and simultaneously acquiring EEG recordings for each of the EEG machines, the method comprising:
monitoring a plurality of EEG machines from a central station, each of the plurality of EEG machines comprising a plurality of electrodes, an amplifier and processor, the central station in communication with each of the plurality of EEG machines over a network, each of the plurality of EEG machines in a separate room;
acquiring EEG signals from each active EEG machine of the plurality of EEG machines;
transmitting the EEG signals over a network to a processing engine at a client device;
processing the EEG signals at the processing engine to generate processed EEG recordings for each of the active EEG machines of the plurality of EEG machines and a plurality of trends for each of the processed EEG recordings;
displaying each of the processed EEG recordings for each of the active EEG machines of the plurality of EEG machines and at least one trend of the plurality of trends in a separate cell of a plurality of separate cells on a display page at the client device;
wherein the processing engine is configured to monitor each channel for electrode artifacts during acquisition of the EEG signals and remove the electrode artifacts to provide a higher level of seizure detection accuracy than without removal of the electrode artifacts;
wherein the processing engine is configured to detect artifacts in the EEG signals by analyzing a plurality of features;
wherein an artifact intensity is illustrated in each of the plurality of separate cells, the artifact intensity shown as a series of horizontal lines comprising a horizontal line for a muscle artifact, a horizontal line for a chewing artifact, a horizontal line for a vertical eye movement artifact, and a horizontal line for a lateral eye movement artifact;
wherein the plurality of trends comprises a seizure probability trend showing a calculated probability of seizure activity over a predetermined time, a rhythmicity spectrogram measuring the amount of rhythmicity which is present at each frequency in an EEG record, a left hemisphere trend, a right hemisphere trend, a fast Fourier transformation (FFT) spectrogram left hemisphere trend, a FFT spectrogram right hemisphere trend, an asymmetry relative spectrogram trend, and an ambulatory EEG (aEEG).

13. The method according to claim 12 further comprising assigning a separate cell of the plurality of separate cells to a newly active EEG machine.

14. The method according to claim 12 further comprising switching between multiple modes in each separate cell of the plurality of separate cells.

15. The method according to claim 12 further comprising updating each separate cell of the plurality of separate cells in real-time.

16. The method according to claim 12 further comprising opening an active EEG recording and occupying the entire display page with a single separate cell of the plurality of separate cells using a one-click function of the client device.

17. The method according to claim 12 further comprising changing a parameter of a plurality of parameters of one separate cell of the plurality of separate cells without affecting the plurality of parameters of any other separate cell of the plurality of separate cells on the display page.

18. The method according to claim 12 further comprising viewing different open EEG recordings on the display page, viewing a partial EEG recording, and permitting a one-click function of the client device to open an active EEG recording and occupy the entire display page with the single separate cell of the plurality of separate cells.
